(19) United States
(12) Patent Application Publication
(10) Pub. No.: US 2024/0118191 A1
(43) Pub. Date: Apr. 11, 2024

(54) METHOD AND APPARATUS FOR STRATIFYING RESPIRATORY INFECTED PATIENTS

(71) Applicant: ILOF - INTELLIGENT LAB ON FIBER, LDA, Matosinhos (PT)

(72) Inventors: Cristiana Raquel DA SILVA CARPINTEIRO, Matosinhos (PT); Joana Isabel SANTOS PAIVA, Matosinhos (PT); Simão Pedro MARQUES PINTO DE FARIA, Matosinhos (PT); Vanessa Patrícia DIAS PINTO, Matosinhos (PT)

(21) Appl. No.: 18/270,252

(22) PCT Filed: Dec. 29, 2021
(86) PCT No.: PCT/EP2021/087808

§ 371 Date: Jun. 29, 2023

(30) Foreign Application Priority Data
Dec. 29, 2020 (LU) LU102350

(51) Int. Cl. G01N 21/31; G01N 33/487; G06N 3/09

(52) U.S. Cl. CPC G01N 21/31; G01N 33/487; G06N 3/09; G01N 2201/06113

(57) ABSTRACT

A method for stratifying a patient infected with a respiratory disease is disclosed. The method comprises providing (1510) a fluid sample (9) from the patient, producing (1520) a light signal from a laser (1), illuminating (1530) the fluid sample (9) with the light signal through a lens in a sensing probe (8), acquiring (1540) a spectrogram from the fluid sample (9), extracting (1550) a plurality of spectrogram features from the light signal, comparing (1560) the extracted plurality of spectrogram features with a model in a database to determine a degree of severity of the respiratory disease. A result is then output (1570) to indicate the degree of severity of the respiratory disease.

### CROSS-REFERENCE TO RELATED APPLICATIONS

This application claims priority of Luxembourg Patent Application number LU102350, filed on 29 Dec. 2020. The entire disclosure of the Luxembourg Patent Application number LU102350 is hereby incorporated herein by reference

### BACKGROUND TO THE INVENTION

The 2019 outbreak of Severe Acute Respiratory Syndrome Coronavirus 2 (SARS-CoV-2) was officially declared by the World Health Organization (WHO) as a global pandemic in March 2020. The worldwide spread of the coronavirus disease (COVID-19) caused, up to date, 34 079 542 confirmed cases and 1 015 963 deaths, reported by WHO [1].

Most of the COVID-19 patients have mild symptoms, such as, fever, cough, fatigue, headache, and shortness of breath [2,3], and can recover at home. However, other severe cases may rapidly progress to severe acute respiratory distress syndrome (ARDS) or develop pneumonia, which may require an immediate and a long-term hospitalization and constant monitoring by health care professionals. In the most severe cases, patients may need a ventilator to support their breathing [2,4]. Unfortunately, the mortality rate of COVID-19 patients that develop pneumonia is considerable high and the survival time, between ICU admission and death, is approximately of 1-2 weeks [4].

The COVID-19 Patients can get pneumonia because of associated viral infections, but also develop co-infections, for instance, pneumonia caused by other microorganisms [5]. Nevertheless, bacteria, for instance, Streplococcus pneumoniae, are usually the cause of Community-Acquired Pneumonia (CAP). However, other viruses, such as the Influenza Virus may also cause a viral CAP [6]. During this COVID-19 pandemic and considering that both viral and bacterial infections are associated with similar symptoms, it has become difficult to differentiate viral COVID-19 pneumonia from pneumonia as a complication from other diseases or from bacterial pneumonia [5].

Recently, the UK-based National Institute for Health and Care Excellence (NICE) published a rapid guideline to help healthcare professionals to differentiate bacterial from viral pneumonia and adequate the treatment to be applied. For instance, a patient with a COVID-19 viral pneumonia may have a history of symptoms for about a week (insidious onset), severe muscle pain, loss of sense of smell, and difficulty breathing but no pleuritic pain. On the other hand, a patient with a bacterial cause of pneumonia rapidly gets sick after few days of symptoms (acute onset), has pleuritic pain and purulent sputum [7]. A severe complication in COVID-19 patients may be also the development of bilateral pneumonia [8-10], while in bacterial pneumonia there is usually a unilateral positive lung infection [11].

The diagnosis of pneumonia is usually made based on clinical features and chest radiography. In hospitalized patients, additional microbiological data, which requires a blood culture, or a sputum culture (sample of mucus), may help identify which microorganism is causing the infection and guide the antibiotic therapy [6,13]. The confirmed diagnosis of COVID-19 is made by Reverse Transcriptase Polymerase Chain Reaction (RT-PCR) using a nasopharyngeal swab but, in suspected COVID-19 cases, a chest Computed Tomography (CT) scan is recommended by the World Health Organization (WHO) if RT-PCR is not available. The WHO also suggests the use of other chest imaging techniques to complement the clinical and laboratory evaluation in COVID-19 diagnosed patients and decide on hospital admission [14]. Indeed, COVID-19 patients have higher chances of getting pneumonia or other hospital acquired infections. In fact, the most severe cases admitted at the ICU are maintained on mechanical ventilation to survive, which increases the probability of acquiring infections. In these specific cases, the early diagnosis of these co-infections is crucial since the detection and characterization of these infections in a timely manner would improve the treatment of the most severe cases [5].

When chest imaging is not accessible, the diagnosis of pneumonia is based on the clinical features and blood biomarkers. The most robust biomarkers for pneumonia detection are procalcitonin (PCT), and C-reactive protein (CRP). PCT levels higher than 0.25 ng/ml and CRP above 20 mg/L are indicators of bacterial infection, being important diagnostic tools but also potential prognosis biomarkers for evaluating severe cases [15]. One study that analyzed the overall accuracy of these biomarkers for diagnosis and prognosis showed that the Area Under the Curve (AUC) of the Receiver Operating Curve (ROC) of a model that included the information from both PCT and CRP (0.92) was significantly higher than one including only the clinical signs and symptoms of patients (0.79) [16].

Different scoring systems have been developed to estimate mortality and help predicting ICU admission rates and infection evolution in pneumonia patients. The most widely used are the Pneumonia Severity Index (PSI) and CURB-65 (based on 5 criteria: confusion symptoms, urea levels, respiratory rate, blood pressure, and the condition of having 65 years of age or older), which are designed to predict the mortality risk in a 30-day time frame. These systems have been highly recommended to determine the need for hospitalization (in addition to clinical judgement) [6,17]. Other severity scores have been used to predict the intensity of inpatient treatment, for instance, the ATS 2001, ATS/IDSA 2007 minor criteria, SCAP, and SMART-COP, which yielded higher sensitivity and specificity than the PSI or CURB-65 [17,18].

Other studies that use statistical and machine learning techniques to predict patient's outcome to the infection were also developed [19,20]. A new classification and regression tree (CART) model was able to predict mortality for CAP patients admitted at ICU with a sensitivity of 73.4%, and specificity of 49.0% [20]. Machine learning approaches have also been applied to guide the diagnosis of pneumonia. Most of the proposed models are based on chest X-ray images, widely used as a diagnosis technique, to help the decision-making process made by clinicians and radiologists [21,22,23,24].

Patients with comorbidities are more likely to develop severe illness and to be admitted into the intensive care unit (ICU). Older adults are more affected with the SARS-CoV-2 infection [3,25,27] and some studies also found a higher prevalence and risk for males when compared with females [2,3]. The most common comorbidities in hospitalized COVID-19 patients are hypertension, cardiovascular diseases, diabetes, and obesity [3,25,26,27,28].

According to the US-based Center for Disease Control and Prevention (CDC) patients that are at higher risk of severe illness from COVID-19 are older adults and patients of any age with cancer, chronic kidney disease, COPD (Chronic Obstructive Pulmonary Disease), immunocompromised state from solid organ transplant, obesity, serious heart conditions, sickle cell disease or type 2 diabetes mellitus. Patients with asthma, cerebrovascular disease, cystic fibrosis, hypertension or high blood pressure, immunocompromised state from blood or bone marrow transplant, immune deficiencies, HIV, use of corticosteroids, or use of other immune weakening medicines, neurologic conditions, liver disease, pregnancy, pulmonary fibrosis, smoking, thalassemia, or type 1 diabetes mellitus might be at increased risk for severe illness from COVID-19 [29].

Together with age and comorbidities, some blood-based biomarkers may be associated with increased risk of death. Several studies reported that, in COVID-19 patients with severe infection, laboratory results consistently showed lower lymphocytes levels (or lymphopenia) [30,31,32,33,34], higher neutrophils count and, consequently, higher neutrophil to lymphocyte ratio (NLR). Regarding lymphopenia, data suggest that the most affected lymphocytes are B cells, T cells and natural-killer (NK) cells, being significantly decreased in the severe groups [31]. In fact, total T cells (CD4+ and CD8+ T cells) are below normal levels in number [31,35]. Additionally, the total T cells that survive after excessive activation remain functionally exhausted [35]. Lower levels of platelets [30, 32], monocytes, eosinophils, and basophils are also observed in severe cases [31].

Currently, there is evidence that some inflammatory markers are also elevated in the blood of COVID-19 patients, as CRP [2,30,32,33], procalcitonin (PCT) [30] and ferritin [30,33] in serum. In a recently published study, CRP, PCT and ferritin are described as relevant infection biomarkers since they are found increased in very severe COVID-19 patients when compared with severe cases, which can be associated with a secondary bacterial infection [30]. Numerous pro-inflammatory molecules, namely interleukin (IL)-6 [31,32,33], IL-8, IL-10, IL-2 receptor (IL-2R) and tumor necrosis factor alpha (TNF-alpha), are also found increased in severe cases, when compared with the non-severe ones [31]. Other studies pointed the lower albumin [30,32] and increased D-dimer [33,36], cardiac troponin [32] and lactate dehydrogenase (LDH) [33,34], as important hallmarks and high-risk factors in COVID-19 severe cases.

In conclusion, there is increasing evidence that older patients with comorbidities are associated with poor disease outcomes. COVID-19 severity may be monitored by the changes in dysregulated biomarkers, focusing on the significant lymphopenia, and increased levels of CRP, IL-6, LDH, and D-dimer.

Currently, a RT-PCR test is required for the detection of SARS-CoV-2 RNA for diagnosing COVID-19. Usually, a respiratory sample is used, as a nasopharyngeal swab. CT scans are supplementary diagnostic tools to confirm the suspected cases [14]. Given the fact that these diagnostic tools may be time consuming taking into consideration the global health burden caused by this pandemic, prediction tools have been developed for helping the medical community to stratify patients and managing hospitalization and healthcare resources. Nevertheless, the rapid spread of publication in COVID-19 may lead to some misinformation. A systematic review made by the COVID-PRECISE group screened the quality of a high number of publications and found that some models may have high risk of bias and overfitting [37].

Still, some of these prognostic models may be fundamental to create tools for helping treatment decisions management. Factors such as age, comorbidities, blood-biomarkers, for example, lymphocyte count, CRP, and chest imaging are among the most frequently included in the current prognostic models [37].

A prediction model which can differentiate severe COVID-19 from non-severe cases in an early stage with a sensitivity of 77.5% and specificity of 78.4% was recently created based on a multicenter study cohort and comorbidities analysis [38]. A deep learning algorithm capable to predict mortality using top 5 predictors/variables from hospitalized patients and achieving a ROC-AUC of 0.968 was also recently proposed [39].

Another deep learning-based model using both clinical and CT imaging data from a single center showed a progression prediction performance ROC AUC of 0.920. This same model was then validated in different cohorts, yielding a ROC-AUC of 0.874 [40]. A CT only based study was able to show a ROC-AUC of 0.87 and 0.86 in differentiating COVID-19 from other pneumonia and COVID-19 from viral pneumonia, respectively [41].

### SUMMARY OF THE INVENTION

This document discloses a method for stratifying a patient infected with a respiratory disease. The method comprises providing a fluid sample from the patient, producing a light signal from a laser, and illuminating the fluid sample with the light signal through a lens in a sensing probe. A spectrogram is acquired a spectrogram from the fluid sample and a plurality of spectrogram features is extracted from the light signal. The method then compares the extracted plurality of spectrogram features with a model in a database to determine a degree of severity of the respiratory disease. A result is then output. This method enables a quick analysis of the respiratory disease and enables the degree of severity to be determined which can improve triage in a hospital and enables medical treatment to be provided to those patients most in need.

The fluid sample is one of a plasma sample or a serum sample. The extracted features are time features and/or frequency derived features. The light can be modulated to improve the results derived from the spectrogram and the features can be extracted over different periods of time.

In a further aspect, the method includes providing demographic features of comorbidities derived from a patient's health record and comparing both the demographic features and the spectrogram features with the model to determine the degree of severity of the respiratory disease. The inclusion of demographic features improves the quality of the output of the result.

A device for stratifying a patient infected with a respiratory disease is also disclosed. The device includes a laser connected through an optical fiber with a sensing probe with a microlens for illuminating a fluid sample from the patient. A detector is present for acquiring a spectrogram from the sample and a computer is adapted to analyze the spectrogram, extract spectrogram features from the spectrogram, compare the extracted spectrogram features with stored features in a model and output a result of the degree of severity of the respiratory disease.

A method for creation of a model for stratifying a patient infected with a respiratory disease is also taught. The creation of the model uses a plurality of spectrogram features extracted from a light signal and a plurality of demographic features from the patient health record. A learning method is applied to an extracted plurality of spectrogram features and the entered plurality of demographic features to correlate the extracted plurality of spectrogram features and the entered plurality of demographic features with the health outcomes to create the model in a database. The learning method is at least one of a supervised learning method, such as a support vector machine, k nearest neighbors, or random forests, an unsupervised learning method, such as clustering algorithms, or a regression model.

In a further aspect, the learning method comprises training a convolutional neural network using the spectrogram features and then training a support vector machine using an output of the trained convolutional neural network and the demographic features.

### DESCRIPTION OF THE FIGURES

FIG. 1 shows a block diagram of the modules and interconnections. Black arrows represent electrical communication, and white for optical path.

FIG. 2 shows an overview of the apparatus

FIG. 3 shows (a) Simple polymeric lens-like tip and (b) Polymeric lens-like tip with a protective structure surrounding it.

FIG. 4 shows a signal processing pipeline.

FIG. 5 shows an example of a spectrogram of the backscattered signal in a 10 second window.

FIG. 6 shows a schematic architecture of a convolutional neural network.

FIG. 7 shows an SVM model stacking approach.

FIG. 8 shows a schematic architecture of a hybrid CNM

FIG. 9 shows a validation ROC curve of the SVM model trained with the patients' health record data

FIG. 10 shows the test ROC curve of the SVM model trained with the patients' health record data.

FIG. 11 shows a validation of the ROC curve of the CNN and the SVM stack model.

FIG. 12 shows the test ROC curve of the CNN and the SVM stack model.

FIG. 13 shows the validation ROC curve of the hybrid CNN.

FIG. 14 shows the test ROC curve of the hybrid CNN.

FIG. 15 shows an outline of the method

FIG. 16 shows an outline of the training system.

### DETAILED DESCRIPTION OF THE INVENTION

The invention will now be described on the basis of the drawings. It will be understood that the embodiments and aspects of the invention described herein are only examples and do not limit the protective scope of the claims in any way. The invention is defined by the claims and their equivalents. It will be understood that features of one aspect or embodiment of the invention can be combined with a feature of a different aspect or aspects and/or embodiments of the invention.

This document will describe two types of experiments which had the objectives of assessing to see whether the system set out in the Applicant's co-pending patent application Nr. LU 102007 filed on 20 Aug. 2020 is capable of differentiating serum/plasma samples collected from healthy controls from the serum/plasma samples collected from patients infected with COVID-19 infected samples infected with the corona virus. The back-scattered signature of serum samples derived from blood samples provided from healthy control donors and COVID-19 infected patients was used to train an AI model to stratify blind samples from unknown subjects and its performance was evaluated (Stratification among COVID-19 infected patients and healthy control patients).

The AI model was trained by associating fingerprints of the serum/plasma samples collected at the time of diagnosis with later clinical outcome within three categories (“Severe symptoms or ICU patients”, “Mild/Light Symptoms or Internment patients or Non-severe symptoms”) to predict the evolution of the infection in terms of severity degree in a two weeks/one-month time window—after the time of the diagnosis. The fingerprints were based on the back-scattered signature of the serum/plasma samples together with any patient-related information regarding comorbidities. The evolution outcome severity of the patient was used to train an AI model. This AI model was used to stratify blind serum/plasma samples and forecast the impact of the infection in the patient over time. In this context, stratification refers to distinguishing between severe and non-severe cases within COVID-19 infected patients.

The dataset used for training the AI model came from the serum/plasma samples collected from 87 different subjects (provided by Centro Hospitalar Universitirio São João—CHUSJ). Basic demographic data, such as age, gender and comorbidities statistics of the sample's donors are shown in Table 1. The prevalence of the comorbidities per groups of diseases are also shown in the Table 1. The most prevalent comorbidities were cardiovascular diseases and associated risk factors (including, for instance, hypertension, which is an important risk factor for a COVID-19 associated poor prognosis), diabetes, immunodeficiency disorders (for example, patients with an immunocompromised immune system caused by a cancer and transplanted patients), kidney diseases, obesity, and respiratory diseases. Parkinson's disease, dementia, depression, dyslipidemia, chronic gastritis, benign prostatic hyperplasia, osteoporosis, diverticulosis, sleep apnea or Traumatic Brain Injury (TBI) were other conditions characterized by this group of infected patients.

TABLE 1
Demographic data, such as gender, age, and comorbidities
of the 87 COVID-19 infected subjects considered for this
study. Values are expressed as mean ± SD or number N (%).

| | | | COMORBIDITIES PER |
|---|---|---|---|
| TYPE | AGE (Y) | GENDER | groups of DISEASEs |
| ICU | 68 ± 12 | 27% Female | 34% Cardiovascular disease |
| | | 73% Male | 14% Diabetes |
| | | | 10% Immunodepressed |
| | | | 8% Respiratory disease |
| | | | 7% Kidney disease |
| | | | 6% Obesity |
| | | | 28% Other diseases |
| INTERNMENT | 67 ± 20 | 52% Female | 28% Cardiovascular disease |
| | | 48% Male | 11% Diabetes |
| | | | 11% Immunodepressed |
| | | | 9% Respiratory disease |
| | | | 4% Kidney disease |
| | | | 2% Obesity |
| | | | 38% Other diseases |
| MILD | 57 ± 23 | 60% Female | 32% Cardiovascular disease |
| | | 40% Male | 9% Diabetes |
| | | | 3% Immunodepressed |
| | | | 0% Respiratory disease |
| | | | 3% Kidney disease |
| | | | 3% Obesity |
| | | | 53% Other diseases |

The serum/plasma samples collected from healthy controls (provided by BioIVT-Burgess Hill, UK) were also included in this study (10 samples from 10 different subjects in total). Demographic data of this subject's subset can be found in Table 2. Values are expressed as mean±standard deviation or percentage (%).

TABLE 2
Demographic data of Healthy controls.

| TYPE | AGE | GENDER |
|---|---|---|
| HEALTHY CONTROLS | 42 ± 13 | 100% Male |

Both COVID-19 serum or plasma samples were used in this study. The serum samples and the plasma samples were processed from whole blood collected at Hospital São João (HSJ), Centro Hospitalar Universitario de S. João, EPE, from the patients that were admitted into the emergency department. The samples were collected at the time of the diagnosis based on a real time (RT)-PCR analysis. For the samples, the severity of the disease was monitored considering each patient and documented in a two-weeks/one-month time frame after the collection time of the samples. The samples from the patients (collected at the time of diagnosis) were then sub-divided into three different groups based on disease evolution: 1) as “ICU patients”, which showed severe symptoms and were hospitalized at ICU, 2) as “Internment” patients, whose symptoms were milder but that also required hospitalization and 3) as “Light symptoms” patients that were asymptomatic or showed minor symptoms and were sent home for recovering and staying at quarantine/isolation. All the samples were stored at −80° C. and, prior to use, thawed on ice and processed into serum (in case of plasma samples). Analyses were conducted using diluted ones of the serum samples in a ratio of 1:2 in a solution of phosphate-buffered saline (1×PBS). Samples from 87 different COVID-19 infected subjects were included in this study.

The serum or plasma samples from individual healthy control donors—BioIVT (Burgess Hill, UK)—were also stored at −80° C. and, prior to use, were thawed on ice. A defibrination procedure was carried out as follows. The plasma samples were thawed on ice and a volume of 4-4,5 μL of [611 U/mL] Thrombin (System Biosciences, CA, USA) was added per 500 μL of plasma to achieve a final concentration of [5 U/mL] Thrombin. The samples were incubated at room temperature (RT) for 5 minutes while gently mixing. The tubes were then centrifuged at 10,000 rpm, for 5 minutes. After centrifuge, a fibrin pellet was noticed, and the supernatant transferred to a new clean tube. The plasma samples already thawed and converted to serum were immediately diluted after the defibrination protocol, while the serum samples were thawed on ice prior the dilution step. All serum samples were analyzed after a 1:2 dilution in 1×PBS.

The optical fiber used in the optical apparatus requires a polymeric lens which is manufactured as follows. The polymeric microstructures used for the lens are fabricated through a guided wave photopolymerization process on top of cleaved optical fibers [25-27], a process in which the cross-linking of monomers is triggered by light with a specific wavelength. Two components must be present in the solution for the photopolymerization process to take place, a monomer, and a photo-initiator. In this non-limited aspect, the monomer was pentaerythritol triacrylate (PETIA) (n=1.48) and the photo-initiator used Bis(2,4,6-trimethylbenzoyl)-phenylphosphineoxide (IRGACURE 819). This photo-initiator is sensitive to wavelengths between 375 nm and 450 nm.

Once the correct proportion between the monomer and the photo-initiator is achieved, an optical setup consisting of a couple of mirrors and a CW laser is used to excite the photo-initiator. In this setup, a laser emitting at a wavelength of 405 nm (Omicron, Rodgay-Dudenhofen, Germany, #Model LuxX cw, 60 mW) is incident at 45° in two consecutive mirrors, resulting in a square shape optical path. After the second reflection, the laser is coupled into an optical fiber by an objective. Since the optical fiber (Thorlabs, Newton, New Jersey, USA #Model SM 980-5.8-125) has a multi-mode behavior for this wavelength, a multitude of optical modes can be excited, resulting in a different optical output pattern and a consequent difference in the geometry imprinted in the tip.

The ideal shape of the polymeric tip structure is a spherical, lens-like termination so that the polymeric tip structure efficiently focuses the incident light. This requires the excitation of a mode with a Gaussian or Gaussian-like profile. Such profiles can be attained with the LP01 and LP02 optical fiber modes. Careful alignment of the setup is required to guarantee the excitement of one of these modes and hence maximum reproducibility.

Once the setup is aligned, i.e., one of the LP01 or LP02 modes is observable at the output of the cleaved fiber, the laser is turned off and the fiber is vertically dipped in a drop of the monomer containing a percentage of photo-initiator between 0.2% to 0.5% in weight. When the fiber is retrieved, a drop of solution stays on the apex of the cleaved fiber, and once the laser is turned on the photopolymerization process occurs. The process is characterized by a self-assembly effect and results in a refractive index increase in the areas on which the laser beam is incident, creating a self-guiding effect that will prevent radiation from scattering to other areas of the drop. A 10-seconds exposure is enough to obtain the desired shape. A long exposure period would result in a flat tip surface and not on the desired mode imprint.

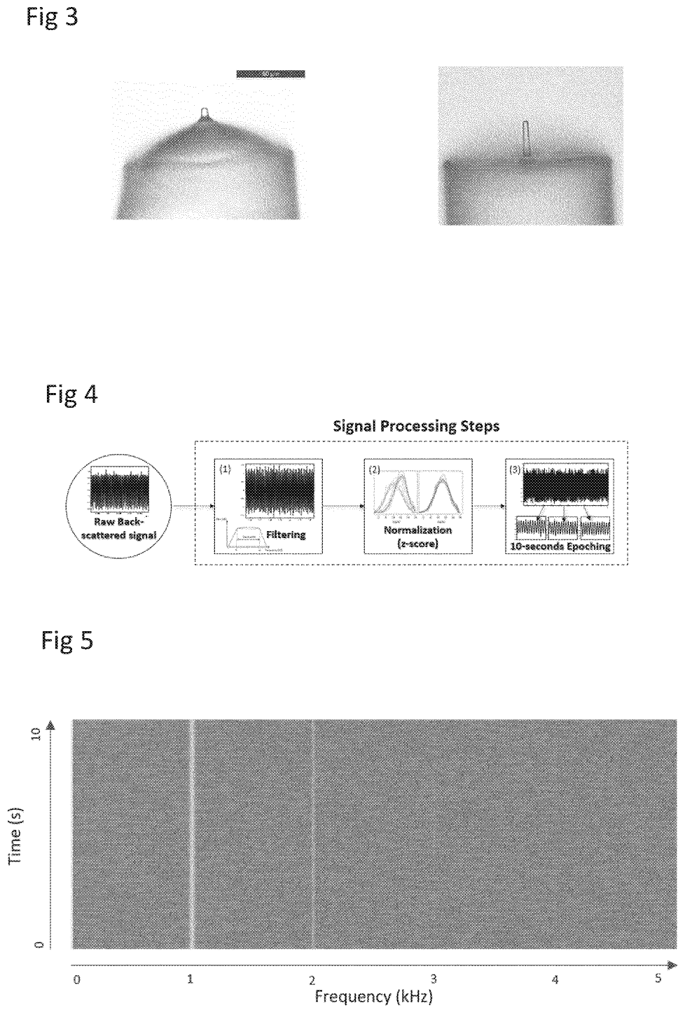

After rinsing the non-polymerized left off polymer with ethanol (96%), the final structure has the diameter of the excited fiber mode and the visual aspect of a spherical lensed tip as depicted in FIG. 3 (a). Given its high aspect ratio (AR), the tip is a very fragile structure by itself. As such, to increase the contact surface and decrease the AR, a protective structure is built around the original tip, assuring a more robust structure. This second step of the fabrication process consists of dipping the already built tip in a new monomer solution containing around 2% of photo-initiator in weight (the same concentration of photo-initiator used for the tip fabrication can also be used in this step). Then a visual verification is conducted to see if the tip's extremity is left outside of the drop. In the cases in which this is verified, the laser is turned on at approximately 20 μW for 3 minutes. When that does not occur naturally, a few drops of ethanol (70-96%) are approximated to the tip, resulting in a rise of the solution drop along the optical fiber, exposing the tip's extremity. Once this is achieved, the exposure proceeds with the same parameters previously mentioned, resulting in a structure like the one presented in FIG. 3 (b).

During the fabrication procedure, some geometrical parameters, such as diameter and length, and the curvature radius of the tip should be controlled. This can be done through the manipulation of some fabrication parameters, such as the optical fiber mode excited during polymerization, as previously mentioned, but also the percentage of the photo-initiator present in the solution, the exposure time, and laser power used during the photo-initiation, etc. To assure a high reproducibility of these tips, these parameters must be left constant throughout the whole fabrication process of a batch of polymeric optical tips. The requirements that must be kept constant as well as the parameters to control are summarized in Table 3.

TABLE 3
Requirements and parameters to control during tip production.

| Requirement | Parameters to control |
|---|---|
| Spherical top | Excited optical mode |
| Similar tip radius for all tips | Laser Power |
| | Exposure Time |
| | Photo-initiator concentration |
| Similar tip length for all tips | Monomer drop left on the fiber |
| | Laser Power |
| Similar refractive index for all tips | Laser Power |
| Similar protective structure | Second monomer drop |
| (Geometry and refractive index) | Laser Power |
| | Exposure Time |
| | Photo-initiator concentration |

For the purposes of the work presented in this document, the fabrication parameters used in the photopolymerization process were the following:
Laser Power (Tip): ≈4 μW
Laser Power (Protection): ≈25 μW
Exposure Time (Tip): 10 s
Exposure Time (Protection): 3 min
Photo-initiator concentration (Tip & Protection): 0.3%

These parameters resulted in the tip structures with lengths ranging from 30 μm to 50 μm, with the basis of the tips having diameters that range from 4 μm to 7 μm, depending on the mode at the fiber's output. Pending on the mode, the curvature radius of the lens structures also varied between the values of 1.5 μm to 3 μm. The numerical apertures (NA) values range between 0.25 and 0.5 (values evaluated in a water medium) and a focused spot with dimensions of about ⅓^{rd} to ¼^{th} of the base diameter of the lens was obtained. The protective structure does not significantly affect the light propagation in the simple tip underneath the protective structure. The protective structure increases the contact area between fiber and polymer to the totality of the optical fiber cross-section, improving the mechanical resistance of the polymeric tip to the successive media crossings to which the polymeric tip will be exposed (e.g., air to plasma, air to serum, etc.). This protective structure has the aspect of a cupula placed around the initial tip, always having a height lower than the tip itself.

It will be appreciated that the probes to apply in this technique are not limited to the polymeric ones described above. Any structure capable of focusing light to a small spot and thus generate an electric field gradient can successfully be used in the method described in this document. Such structure can be built on the apex or on the side of an optical fiber or on a planar substrate. These structures include optical fiber tapers, phase Fresnel plates (fiber or planar), a single nanometric hole, or an array of nanometric holes on a metallic surface, for plasmonic effects. The latter can either be deposited on an optical fiber or on a transparent planar substrate. To summarize, any type of metalens, be it metallic or dielectric, built on an optical fiber or on a planar substrate is suitable for this application.

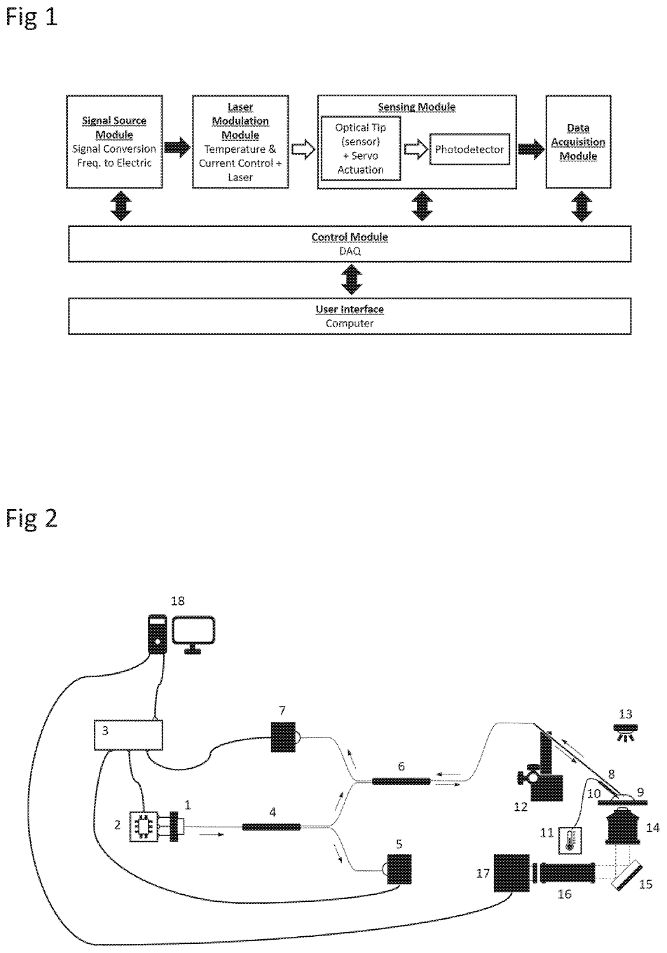

The setup used for acquiring the back-scattered signal from the liquid dispersion samples using the spherical-lensed optical fiber tip was composed by the following modules shown in FIG. 1. Black arrows represent electrical communication, and white for optical path.

FIG. 1 shows a sensing module 10 which comprises the lensed optical fiber (the sensing probe) inserted into a metallic capillary and manipulated using a 4-axis micromanipulator, two silicon photodetectors, and a Type-T thermocouple with logger. A laser module 20 comprises the light source (976 nm diode laser) and corresponding submodules for laser temperature and current control. A data acquisition module 30 is composed by a Data Acquisition board (DAQ). A visualization and imaging system 40 include the optical components needed to visualize the optical fiber tip at the micro-scale. A control unit 50 includes software, hardware controlling and recording and processing the acquired data (the back-scattered signal, the signal collected at the output of the laser and the obtained images).

A detailed schematic of the data acquisition apparatus is depicted in FIG. 2. It will be appreciated that this is only one example of the data acquisition apparatus for obtaining the data to establish the AI model and that other apparatus are possible.

The irradiation laser (1: Lumentum Operations LLC, San Jose, CA, Catalog #S28-7602-500), emitting at 976 nm wavelength, was modulated in frequency by a sinusoidal signal (fundamental frequency of 1 kHz, to escape from the electrical grid 50 Hz harmonics) digitally generated at a sampling rate of 10 kHz using a custom-build MATLAB script according to the equation:

1.45+0.045*sin(2*π*1000*t), t=time in seconds

so that, considering the laser driver's gain, the laser characteristic curve, and the optical loss along the fiber components, the lens' maximal output optical power was 40 mW. This value was determined in accordance with the values used in the literature for optical delivery, collection and manipulation effects through optical fibers considering the selected wavelength value range, and to cause as little damage as possible to the biological human-derived samples [28].

The modulation signal was externally injected into the laser driver (2: MWTechnologies Lda, Portugal, Model #cLDD) through one of the output digital-to-analog ports of the data acquisition board (3: NI, Austin, TX, Model #USB-6212 BNC). The resulting optical signal, mirroring the modulation equation, is inserted into the optical fiber and passes through a 1/99 optical coupler (4: Laser Components GmbH, Germany, Model #3044214). While most of the radiation follows to the rest of the optical circuit, 1% of the radiation is monitored using a silicon photodetector (5: Thorlabs Inc, Newton, NJ, Model #PDA-36A2) connected to one DAQ analog-input port.

The modulation signal was calculated by a modulation equation which was dependent on one or more of the gain of the laser driver 2, the characteristic of the laser and the optical loss along the optical fiber. The modulation signal was chosen to reduce power losses along the optical path from the laser to fiber tip and thus increase the energy of the signal reflected by any particles in the blood or serum samples. This enabled the identification of particles in the 70-110 nm range, which appears to be typical of the corona virus, as reported by Kim, J. M., Chung, Y. S., et al. (2020). “Identification of Coronavirus Isolated from a Patient in Korea with COVID-19”, Osong public health and research perspectives, 11(1): 3-7. https://doi.org/10.24171/j.phrp.2020.11.1.02, Prasad, S., Potdar, V., et al. (2020). “Transmission electron microscopy imaging of SARS-CoV-2”, The Indian journal of medical research, 151(2 & 3): 241-243. https://doi.org/10.4103/ijmr.IJMR_577_20, or Menter, T., et al. (2020), “Postmortem examination of COVID-19 patients reveals diffuse alveolar damage with severe capillary congestion and variegated findings in lungs and other organs suggesting vascular dysfunction”, Histopathology, 77(2), 198-209. https://doi.org/10.1111/his.14134.

The data acquisition device should also be able to detect other types of viruses, such as influenza viruses, adenoviruses, human metapneumovirus (hMPV) or respiratory syncytial virus (RSV). For example, human parainfluenza viruses have an average size of 150 nm, as reported by Henrickson K. J. (2003). Parainfluenza viruses. Clinical microbiology reviews, 16(2): 242-264. https://doi.org/10.1128/CMR.16.2.242-264.2003. Influenza A viruses are reported to be 80 to 120 nm in diameter by Noda, T., et al (2006). Architecture of ribonucleoprotein complexes in influenza A virus particles. Nature 439: 490-492. https://doi.org/10.1038/nature04378. Influenza A and B viruses are very identical and the spherical forms of both have around 100 nm in diameter, while the filamentous forms above 300 nm in length, as discussed in Bouvier, N. M., & Palese, P. (2008). The biology of influenza viruses. Vaccine, 26 Suppl 4(Suppl 4), D49-D53. https://doi.org/10.1016/j.vaccine.2008.07.039.

Adenoviruses virions are known to range in size from 70 to 100 nm (see Doerfler W (1996). Adenoviruses. In: Baron S, editor. Medical Microbiology. 4^{th} edition. Galveston (TX): University of Texas Medical Branch at Galveston; Chapter 67. https://www.ncbi.nlm.nih.gov/books/NBK8503/ and Kennedy, M. A., & Parks, R. J. (2009). Adenovirus virion stability and the viral genome: size matters. Molecular therapy: the journal of the American Society of Gene Therapy, 17(10): 1664-1666. https://doi.org/10.1038/mt.2009.202). Human metapneumovirus (hMPV) particles were found in the range of 150 to 600 nm in size (see van den Hoogen, B. G. et al. (2001). A newly discovered human pneumovirus isolated from young children with respiratory tract disease. Nature medicine, 7(6): 719-724. https://doi.org/10.1038/89098). Respiratory syncytial virus (RSV) is also variable in shape, but the average diameter of both spherical and filamentous forms is, approximately and respectively, 150 nm and 100 to 120 nm (T Bächi; Direct observation of the budding and fusion of an enveloped virus by video microscopy of viable cells. J Cell Biol 1 Nov. 1988; 107 (5): 1689-1695. https://doi.org/10.1083/jcb.107.5.1689, Reena Ghildyal, Adeline Ho, David A. Jans, (2006). Central role of the respiratory syncytial virus matrix protein in infection, FEMS Microbiology Reviews, 30(5): 692-705. https://doi.org/10.1111/j.1574-6976.2006.00025.x and Griffiths, C., Drews, S. J., & Marchant, D. J. (2017). Respiratory Syncytial Virus: Infection, Detection, and New Options for Prevention and Treatment. Clinical microbiology reviews, 30(1), 277-319. https://doi.org/10.1128/CMR.00010-16).

A 50/50, 1×2, optical coupler (6: AFW Technologies Pty Ltd, Australia, Model #FOSC-1-98-50-L-1-H64F-2) establishes a bidirectional connection between the incoming light from the laser module, the sensing photodetector (7: Thorlabs Inc, Newton, NJ, Model #PDA-36A2) and the sensing probe (8: the microlensed optical fiber with its end just outside a metal capillary). This allows the sensing probe to simultaneously focus the light coming from the laser and the collection of the back-scattered radiation arising from the liquid dispersion sample (9) to be analyzed. To provide further information about the samples' conditions/properties, temperature readings are obtained using a Type-T Thermocouple (10: Omega Engineering Ltd, Manchester, UK, Model #TC-TT-TI-24-2M), connected to a USB data logger (11: Omega Engineering Ltd, Manchester, UK, Model #OM-EL-USB-TC-LCD).

By controlling the temperature of the sample and the laser output, it was also possible to correct signal artifacts correlated with particles diffusion coefficient's behaviour increasing our signal-to-noise ratio and, consequently, distinguish particles with different sizes with a narrower range within our spectrum.

The sensing probe 8 is manipulated using a 4 axis (x, y, z, and tilt) right-hand micromanipulator (12: Siskiyou Corporation, Grants Pass, OR, Model #:MX7600) with a probe holder where the capillary is fixed. This manipulator is connected to a closed-loop dial controller (Siskiyou Corporation, Grants Pass, OR, Model #:MC1000e-R1/4T) that allows a more precise displacement of the probe into and inside the sample.

The visualization and imaging module is composed by a self-made inverted microscope setup using a standard white LED light source (13), an objective (14, currently at 20×, but higher amplification can be used to observe smaller volumes), a mirror (15) and a zoom lens (16: Edmund Optics, Barrington, NJ, Model #VZM 450). This microscope drives the desired imaging plane to a digital camera (17, Edmund Optics, Barrington, NJ, USA Model EO-1312C #Catalog 83-770). The image is observed in real-time in the lab's computer (18) using IDS:'s software uEye Cockpit. The camera's sensing region allows for the visualization of the focused infrared beam and its reaction with the sample's constituents.

To prevent cross-contamination between samples, a standard cleaning protocol was followed. The sensing probe 8 was inserted into a solvent (e.g., diluted bleach) between any two samples to remove any biological traces. Then, the sensing probe 8 was dipped in distilled water to remove any trace of bleach. While in the water, one to two signal acquisitions (as above) were performed to ascertain any degradation issues and ensure probe prime conditions.

Before the feature extraction, the back-scattered signal was pre-processed. After that, a set of 98 features were calculated for each 10 second time window (and these features are shown in Table 3). These features can be divided into two types: time and frequency derived. Within the time domain features it is possible to group them into time domain metrics and non-linear. On the other hand, frequency related features can be subdivided in wavelet packet decomposition, DCT-derived and spectral features. The feature extraction step was implemented with a custom-built python 3 script, using the scipy, pandas, PyWavelets, librosa and, numba python libraries. It will be appreciated that the set of 98 features is not limiting of the invention and that fewer features can be extracted or more features could be developed.

One or more of the following features were extracted.

| Type | Group | Feature |
|---|---|---|
| Time- | Time | Standard Deviation |
| domain | domain | Interquartile range |
| | metrics | Kurtosis |
| | | Skewness |
| | | Mean |
| | | Root mean square |
| | | Signal power |
| | | Entropy |
| | | Root sum of squares level |
| | | Area under the curve histogram |
| | Non- | Approximate entropy |
| | linear | Singular value decomposition entropy |
| | | Petrosian fractal dimension |
| | | Higuchi fractal dimension |
| | | Detrended fluctuation analysis coefficient |
| | | Hurst Exponent |
| | | Hjorth complexity |
| | | Hjorth mobility |
| Frequency- | DCT- | 1^{st} to 30^{th} DCT coefficients (30) |
| domain | derived | Number of DCT coefficients that capture 98% |
| | | of the original signal |
| | | Total spectrum Area Under Curve |
| | | Spectral Entropy |
| | | 1^{st} to 10^{th} Hilbert peaks (10) |
| | | Number of Hilbert coefficients that capture 98% |
| | | of the original signal |
| | | Haar Relative Power of the first 6 levels (6) |
| | | Db10 Relative Power of the first 6 levels (6) |
| | | Symlet Relative Power of the first 6 levels (6) |
| | | Db4 Relative Power of the first 6 levels (6) |
| | Spectral | Spectral contrast std |
| | | Spectral contrast mean |
| | | Spectral contrast max |
| | | Spectral roll-off frequency std |
| | | Spectral roll-off frequency mean |
| | | Spectral roll-off frequency max |
| | | Spectral flatness std |
| | | Spectral flatness mean |
| | | Spectra flatness max |
| | | Spectral centroid std |
| | | Spectral centroid mean |
| | | Spectra centroid max |

For the extraction of all numeric features, the back-scattered signals were first pre-processed using the pipeline schematized in FIG. 4. These steps were applied to each raw signal acquisition set, before extracting the features which characterize the samples and applying any learning method. A custom-built Python 3 script was created for running this pipeline, using the numpy and scipy libraries.

Each acquisition was first filtered using a second-order 500 Hz Butterworth high-pass filter to remove noisy low-frequency components of the acquired signal (e.g., 50 Hz electrical grid component). Then, the signal of each acquisition was normalized using the z-score. The z-score can be calculated using the following equation:

$$z = \frac{x - mean ( x )}{SD ( x )}$$

where mean(x) and SD(x) represent, respectively, the signal average and standard deviation. After this transformation, each whole acquisition was split into time windows of 10 seconds. Features were calculated for each 10 second time window.

Time domain metrics such as mean, standard deviation, root mean square, signal power, root sum of squares level (RSSQ), skewness, kurtosis, interquartile range, and entropy were used, given its adequacy in differentiating types of periodic signals. The skewness reflects the distribution symmetry degree while kurtosis quantifies whether the shape of the data distribution matches the Gaussian distribution. The interquartile range is a variability measure. Additionally, the area under the curve of the histogram distribution of the voltage values was considered.

Non-linear features are useful to describe the complexity and regularity of a signal and are often used to describe the phase behavior of predominantly stochastic signals, such as EEG. A total of 8 non-linear features were considered: approximate entropy, singular value decomposition (SVD) entropy, Petrosian fractal dimension, Hurst exponent, Detrended fluctuation analysis (DFA), Higuchi fractal dimension, Hjorth complexity and mobility. The approximate entropy is used to quantify the amount of regularity and the unpredictability of fluctuations over time-series data, whereas the SVD entropy is an indicator of the number of eigenvectors that are necessary for an adequate explanation of the data set, in other words, it measures the dimensionality of the data.

The term fractal relates to fluctuations in time that possess a form of self-similarity whose dimension cannot be described by an integer value. Therefore, a fractal dimension (FD) is a ratio that provides a statistical index of complexity and the degree of irregularity of a waveform. It is a highly sensitive measure for the detection of hidden information contained in physiological time series. Petrosian's algorithm provides a fast computation of the FD of a signal by translating the series into a binary sequence, while Higuchi is iterative in nature and is especially useful to handle waveforms as objects. Finally, DFA is a method for quantifying fractal scaling and correlation properties in the time-series.

The Hurst exponent is a measure of the “long-term memory” of a time series. It can be used to determine whether the time series is more, less, or equally likely to increase if it has increased in previous steps. Hjorth parameters are indicators of the statistical properties of a signal in the time domain. The mobility parameter is defined as the square root of the ratio of the variance of the first derivative of the signal and that of the signal y(t):

$$Mobility = \sqrt{\frac{var ( \frac{dy ( t )}{dt} )}{var ( y ( t ) )}} \qquad Eqn . ( 1 )$$

The mobility parameter represents the mean frequency or the proportion of standard deviation of the power spectrum.

On the other hand, the complexity parameter indicates how the shape of a signal is like a pure sine wave, this value converges to 1 as the shape of the signal gets more similar to a pure sine wave. The complexity parameter is defined by the following expression:

$$Complexity = \frac{Mobility ( \frac{dy ( t )}{dt} )}{Mobility ( y ( t ) )} \qquad Eqn . ( 2 )$$

Regarding the frequency-domain analysis of the back-scattered signal, three sets of features were extracted: Discrete Cosine Transform (DCT) parameters, Wavelet derived coefficients and spectral features. The DCT was applied to each time window. The DCT can capture minimal periodicities of the signal, without injecting high-frequency artifacts in the transformed data. Besides being highly adequate to short signals, it is highly attractive for this type of problems which require to differentiate target classes, because DCT coefficients are uncorrelated. Thus, they can be used as suitable features for characterizing each peptide class. Additionally, the DCT can embed most of the signal energy into a small number of coefficients. The first n coefficients of the DCT of the scattering echo signal are defined by the following equation:

$${E}_{i}^{D C T} [ l ] = {\sum }_{\dot{κ} = 0}^{N - 1} {ε}_{i} [ k ] cos [ \frac{π l ( 2 k + 1 )}{2 N} ] , for l = 1 , … , n$$

where εᵢ is the signal envelope estimated using the Hilbert transform. The following features were extracted from DCT analysis: the number of coefficients needed to represent about 98% of the total energy of the original signal, the first 30 DCT coefficients, the Area Under the Curve (AUC) of the DCT spectrum for all the frequencies before the modulation frequency (1 kHz) and, the entropy of the DCT spectrum. A similar analysis was conducted using the Hilbert transform. The Hilbert transform when applied to the signal produces an analytical real-valued representation of it. The 10 highest amplitude peaks of the Hilbert transformed signal were used as features, as well as the number of coefficients needed to represent about 98% of the total energy of the original signal.

Some parameters based on the information extracted from wavelet analysis of each original signal portion were also considered as features. Using wavelet packet decomposition, it is possible to extract, in each frequency band, certain tonal information of the original signal depending on the frequency range and content of the back-scattered signal. For this process, it is necessary to choose a suitable mother wavelet, that will be used as a prototype to be compared with the original signal and extract frequency sub band information. Four mother wavelets—Haar, Daubechies (db10 and db4) and Symlet—were selected to characterize the backscattered signal portions. Six features for each type of mother Wavelet based on the relative power of the Wavelet packet-derived reconstructed signal (one to six levels) were considered.

Spectral features characterize the signal's power spectrum, which is the distribution of power across the frequency components composing that signal. It is obtained using the Fourier Transform. Four measures were derived from the spectrum: spectral flatness, spectral centroid, spectral contrast and spectral roll-off. A total of 12 features were calculated from these measures. The spectral contrast is defined as the difference between valleys and peaks in a spectrum. For each sub-band, the energy contrast is estimated by comparing the mean energy in the top quantile (peak energy) to that of the bottom quantile (valley energy). The spectral flatness (or tonality coefficient) quantifies the degree to which a signal is noise-like a signal is. A high spectral flatness (closer to 1.0) indicates that the spectrum is like white noise. The spectral roll-off frequency is defined as the center frequency for a spectrogram bin such that at least 85% of the energy of the spectrum is contained in this bin and in the bins below. Finally, the spectral centroid indicates where the center of mass of each frequency bin in the spectrogram is located. For each one of these measures three features were calculated: the mean, the maximum, and the standard deviation.

The subject's metadata was encoded into eight demographic features (as shown in table 5)—the patient's age, the total number of reported comorbidities and six binary features descriptive of the patient's health record. These features were designed to accommodate the most conditioning factors for COVID-19 disease. It will be appreciated that it is possible to use a subset of the demographic features and that other demographic features may be used.

TABLE 5
Demographic Features derived from the reported comorbidities.
Subject's health record features

Age
Total number of comorbidities
Health report not available
Type of comorbidities
Respiratory disease
Diabetes
Kidney disease
Cardiovascular disease
Immunodepression

A spectrogram is a visual representation of the signal's frequency spectrum as the frequency spectrum varies with time. A total of 5 spectrograms was calculated for each 30 second acquisition in this example, but this is not limiting of the invention. The spectrogram captures the behavior of a 10 second window. The windows have an overlap of 5 seconds. The spectrograms were calculated using the Fast Fourier Transform (FFT). The signal was broken up into NFFT segments, overlapping in N overlap points. The FFT was then used to calculate the magnitude of the frequency spectrum for each part. Each segment corresponds to a vertical line in the image—a measurement of magnitude versus frequency for a specific moment in time. These spectrums are finally stacked to form the image. In this non-limiting example, the NFFT was set to 1024 and the N overlap to 512, which results in a 513 by 194 spectrogram. Each pixel encodes information for a 10 Hz frequency interval and a 50-millisecond time interval. An example of a spectrogram can be observed in FIG. 5. The second-most prominent line, after the 0 Hz line, represents the modulation frequency—1 kHz. The harmonic frequencies of the modulated signal can also be observed at N×1 kHz.

The spectrogram allows the use of 2D convolutions that correlate in both the time and frequency domains and is used in the application of the deep learning strategies set out in this disclosure. The combination of this type of data representation (that allowed the transformation of the information from 1D to 2D) and the application of deep learning methods unlock several attributes that were only possible to extract in the 2D domain and increase by N² the quantity of information about the particles analyzed.

Temperature Sensing Based on Back-Scattered Frequency Features

The relationship between the temperature and the frequency features was studied by calculating the correlation between the temporal evolution of the features and the temperature variation throughout the experiment. Correlation values were calculated considering the average temperature between the sample's initial and final temperatures along each acquisition. Similarly, the mean value of each feature was calculated for each acquisition, so that the two time-series to be compared (temperature and each light scattered-derived feature) had the same number of points. The correlation was calculated using the following formula:

$${r}_{xy} = \sum \frac{( {x}_{i} - mean ( x ) ) ( {y}_{i} - mean ( y ) )}{\sqrt{\sum {( {x}_{i} - mean ( x ) )}^{2} {( {y}_{i} - mean ( y ) )}^{2}}}$$

Where xᵢ represents the temperature time-series values and yᵢ the feature values. Each time-series was normalized so that the correlation value lies between 0 and 1.

Four different models for the AI algorithms used for COVID-19 patient stratification were developed. The architecture of conventional algorithms such as the SVM or CNN had to be changed to match the different types of generated data (image, numeric and metadata). In addition to these two models, two additional architectures that modify and combine these two models were designed. The models used in each stratification task varied depending on the type and quantity of data available.

Support Vector Machine (SVM)

Support Vector machines can deal with either linear or non-linear input data, which makes the SVMs suitable for high-dimensionality problems. In a nutshell, the SVM can distinguish between two different groups of data points by finding a separating hyperplane with the maximal margin between the groups (also called classes). Three general attributes define the SVM classifier: C—a hyper-parameter which controls the trade-off between margin maximization and error minimization, the kernel—a function that maps the training data into a high-dimensional feature space and, the sigma, which controls the size of the kernel. The type of kernel function used is a factor on the performance of SVM classification algorithm. The kernel function implicitly maps non-linear features into a high-dimensional features space, where the kernel function can then use linear approaches for solving learning and estimation issues. The types of the kernel function most frequently used are the linear and the Gaussian, or more commonly known as Radial Basis Function SVM (RBF SVM). However, by selecting the RBF kernel function, a third parameter must be optimized: sigma (i.e., the width of the Gaussian function). Larger values of the attribute C are associated to a smaller margin, if the decision function is better at classifying all training points correctly classifier. A lower value of the attribute C encourages a larger margin, therefore a simpler decision function and a less complex classifier. The margin corresponds to the separation between the different classes. A smaller margin means a smaller separation difference between the classes and a larger margin means a large difference between the classes. In the case of sigma (RBF SVM), if sigma is large, the effect of the attribute C becomes negligible. If sigma is small, the attribute C affects the model in the same way as it affects a linear kernel. Several combinations of these parameters were tested to find the optimal model. The reason for this is that, for high values of sigma, the data points need to be very close to each other to be considered in the same group (or class). As a result, high values of gamma typically produce highly flexed decision boundaries, and low values of gamma often results in a decision boundary that is more linear. When signal is large, the decision function is too far from being a linear one and C becomes negligible.

A Convolutional Neural Network (CNN) is a deep learning algorithm commonly used in image analysis. The CNNs distinguish themselves from conventional classification algorithms by their ability to automatically extract the most important features from an image. The algorithm can assign importance (learnable weights and biases) to various aspects of the images and use them to differentiate between image classes. A CNN consists of an input layer, multiple hidden layers, and an output layer. The hidden layers of a CNN typically consist of a series of convolutional layers. These layers will compute the output of neurons that are connected to local regions in the input, each computing a dot product between their weights and a small region they are connected to in the input.

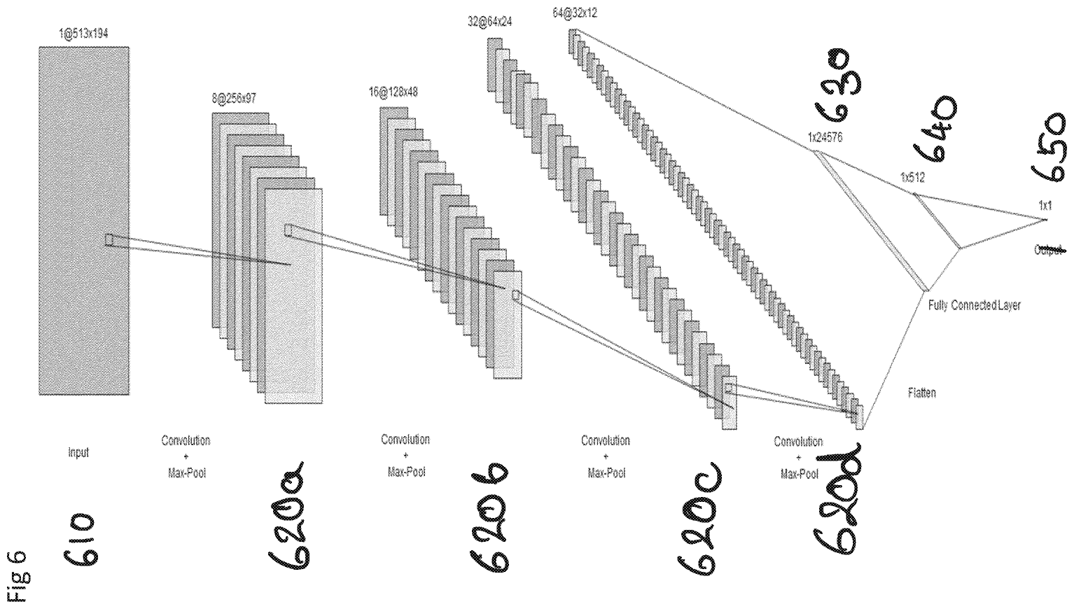

The architecture implemented can be observed in FIG. 6. Each different block in FIG. 6 represents the data shape that interacted with the neuronal layers, the convolutional filters sizes are also represented between each encoding block. The decoding layers are fully connected. The CNN used in this method is based on a single input layer, four encoding blocks, one fully connected layer, and the output, but this is not limiting of the invention.

The first layer 610 is the input layer, which holds the raw pixel values of the spectrogram image, i.e., the spectrogram features. Each encoding block 620a-620d is composed by a convolutional layer followed by an activation layer, a pooling layer, and a dropout layer. The convolution layer that uses multiple filters can extract features from the image dataset while preserving spatial information. After that, the activation layer applies an elementwise ReLU (Rectified Linear Unit) activation function. The ReLU is half rectified, which means that the output f(z) of the function is zero when an input z is negative, and the output f(z) is equal to the input z when z is higher or equal to zero.

The pooling layer follows subsequently. The pooling operation, also called subsampling, is used to reduce the dimensionality of the feature maps resulting from the convolution operation. Pooling is performed using the max-pooling method, which calculates the maximum value for each patch of the feature map. This operation was performed with a 2×2 filter in all encoding blocks. Consequently, the pooling layer will reduce the size of each feature map by a factor of 2, reducing the number of pixels or values in each feature map to one quarter of the size. After that, there is the dropout layer. The dropout layer randomly sets a percentage of input units to 0 at each step during the training time, which helps prevent overfitting.

In the first encoding block 620a, convolution was performed using a kernel size of 5×5 and eight filters in total. In the second encoding block 620b, the same kernel was used, but the number of filters doubled (16 filters in total). The kernel size applied in the third and fourth encoding blocks 620c and 620d was 3×3, while the number of filters was changed to 32 and 64, respectively. The output of the fourth encoding block 620d with a shape of 32×12×64 was then flattened to a 1×24576 tensor 630. Finally, there is the fully connected layer 640, which, as the name suggests, connects every neuron in one layer to every activation unit of the next layer. This layer compiles the data extracted by the previous layers and, after passing through a sigmoid activation function, the layer outputs in 650 the final classification probability.

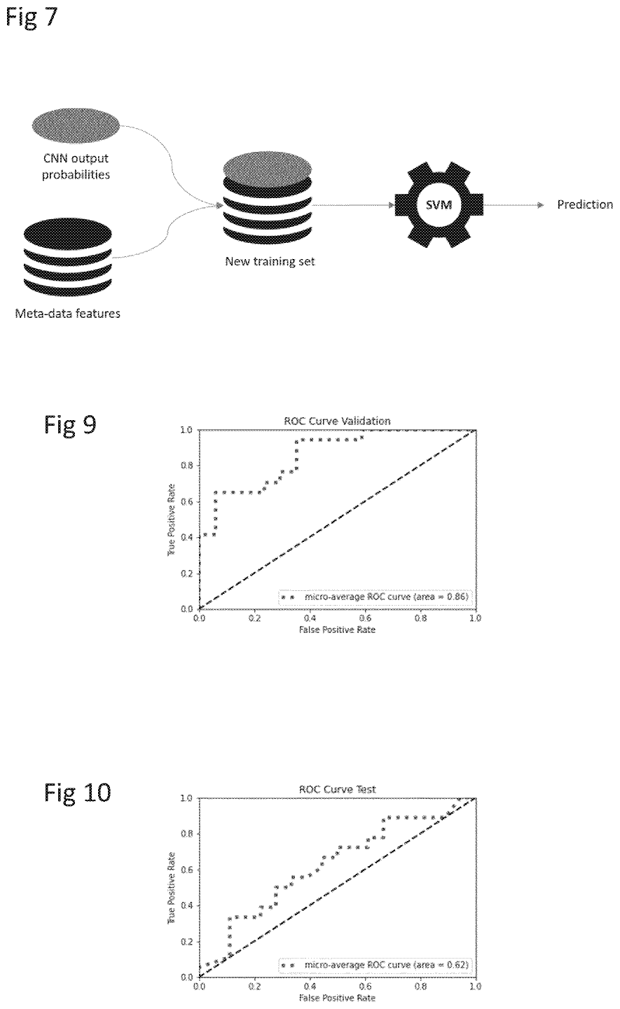

In addition to the above SVM- and CNN-based models, an alternative architecture that combines the two models was built. This combination was developed by creating a new dataset, which results from the aggregation of the CNN output probability 710 (from FIG. 6) with the metadata features 720, as depicted in FIG. 7. The CNN output probability 710 was obtained after independently training the existing CNN model using the spectrogram features and optimizing the existing CNN model using the validation set. The new dataset was then used to train an SVM 730. The SVM 730 was optimized based on the performance using the same validation set as the CNN model used to obtain the CNN output probability 710. This alternative architecture allows for the combination of the information collected by CNN with other types of data.

Construction of a hybrid CNN. The architecture of the CNN was adjusted to combine the features extracted from the spectrogram with both non-spectral and metadata features. This novel architecture can be observed in FIG. 9. The structure stays essentially the same as the one shown in FIG. 6 up until the fourth encoding block 820d (equivalent to 620d). The output of this fourth encoding block 820d—the spectrogram features extracted from the spectrogram—are fed to a fully connected block 830. Simultaneously the non-spectral feature sets 840 and the metadata feature sets 850 are read and the non-spectral feature sets 840 and the metadata feature sets 850 passes through a fully connected layer 860. After that, the output of these three fully connected layers is concatenated into a single layer 870. This new tensor goes through a dense layer 880 and finally, the prediction is made at the output 890.

Stratification between COVID-19 infected patients and healthy control patients. The model used for this stratification task was chosen to consider the dataset constraints. The dataset was composed by samples of 10 healthy subjects and the same number of samples of COVID-19 infected patients. A simple SVM model was used to perform the classification task. The model was trained using a cross-validation strategy. The cross-validation strategy is used to obtain performance values and choose the most suitable model. This cross-validation strategy involves partitioning the data into several subsets. The SVM model is trained used a first one of the subsets (called the training set) and subsequently validated using the other subset (called the validation set). To reduce variability of the model, multiple rounds of the cross-validation are performed using the same initial dataset with different partitions, and the validation results are combined (e.g., averaged) over the rounds to give an estimate of the model's predictive performance. The overall performance of the model is calculated as the average ROC AUC across all validation folders and the optimal model was chosen based on the ROC (Receive Operating Curve) area under the curve (=AUC) across all validation folders.

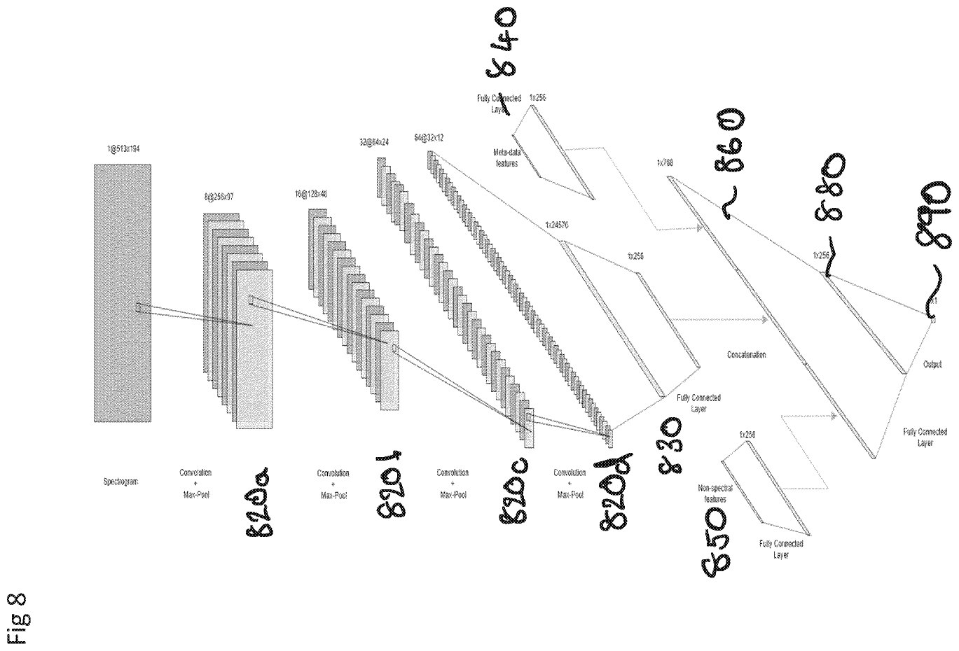

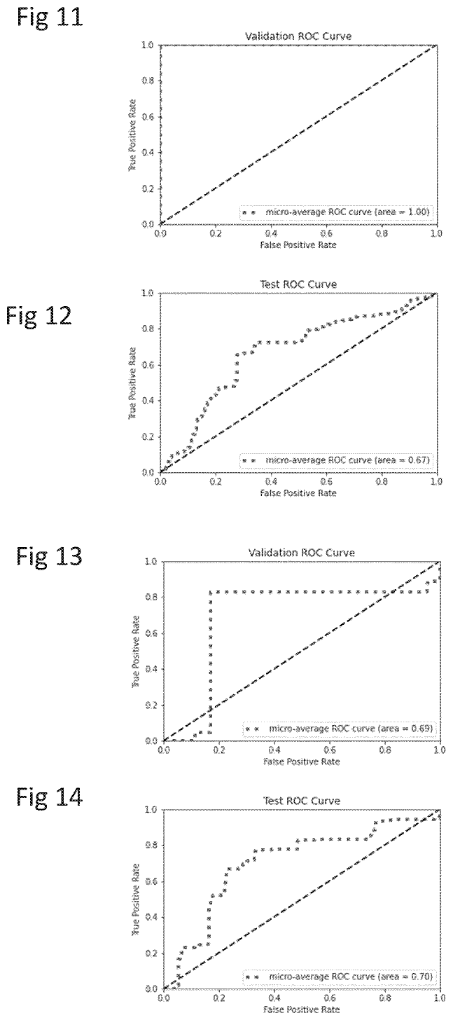

Stratification among COVID-19 infected patients—prediction of the disease evolution. The dataset used to predict COVID-19 patient's evolution was significantly larger, 87 patients, being then possible to apply deep learning approaches—the two CNN-based architectures shown in FIGS. 6 and 8. Additionally, a simpler model, the SVM, was built using only the patients' metadata. The dataset was divided into three parts—Train, Validation, and Test (completely independent from the validation and test set)—by a proportion of 60%, 20%, and 20% of the data from the 87 patients, respectively. The split was made to maintain the label proportions between them, meaning that the three parts of the datasets were composed roughly by the same samples' number of each class (“UCI/severe cases” and “Non-UCI/non-severe cases”).

The training data were used as an input to the models in a way that the models could be adjusted to the data. This is discussed further with reference to FIG. 16. The validation set (the first set of data that was completely blind to the model) was used to select the most suitable model between all the trained models with different configurations. The test set was maintained completely apart from the other set till being used for performance evaluation of the model.

Results

Stratification between COVID-19 infected patients from healthy control patients

The performance results regarding this stratification task are depicted in table 6.

TABLE 6
Performance of the COVID-19 detection model
during the cross-validation stage.

| | Training | Validation |
|---|---|---|
| ROC AUC | 0.94 ± 0.01 | 0.75 ± 0.07 |
| Accuracy | 0.85 ± 0.02 | 0.64 ± 0.04 |

The mean training ROC AUC and accuracy across all the validation folders suggests that the model was able to detect and learn differences between the optical fingerprints of the two classes (healthy controls versus infected patients). However, the drop in performance during validation (column 2 of data) suggests that the model overfitted the training set, which may be explained by the smaller dataset size.

Stratification among COVID-19 infected patients—prediction of the disease evolution.

Metadata SVM. The validation and test ROC curves corresponding to the SVM trained with the patients' metadata are depicted in FIGS. 9 and 10, respectively. The model achieved a ROC AUC of 0.86 in the validation set, but its performance decreased in the test set. The f1-score decreased as well in the test set (table 7) corroborating this fact.

| | Validation | Test |
|---|---|---|
| Accuracy | 0.82 | 0.61 |
| F1-score | 0.82 | 0.53 |
| ROC AUC | 0.86 | 0.62 |

CNN and SVM Stack Model

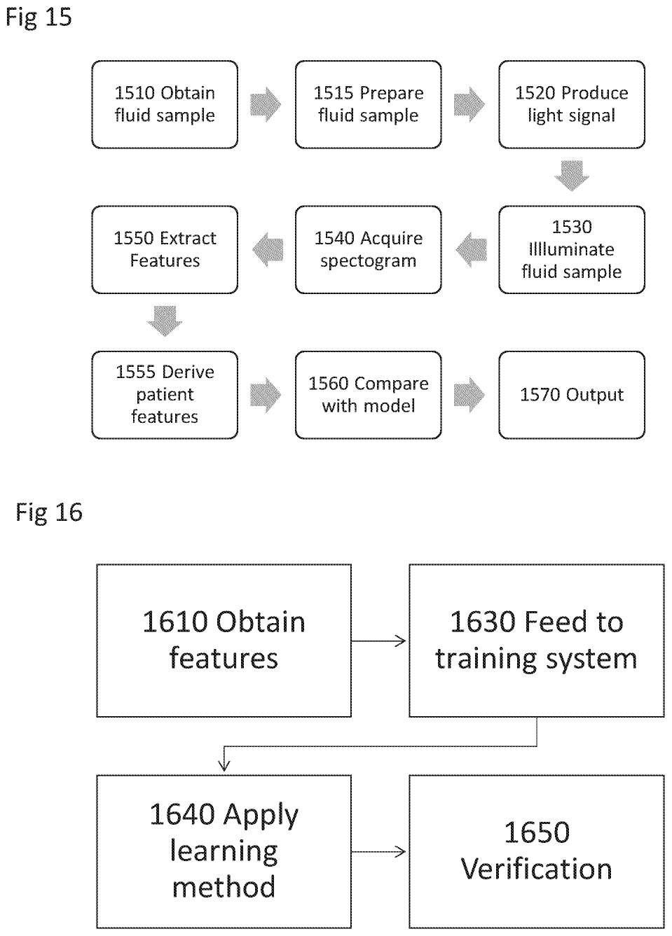

The validation and test ROC curves of the CNN and SVM stack architecture can be depicted in FIGS. 14 and 15, respectively.

The validation ROC curve obtained to this model was a perfect ROC curve—ROC AUC equal to 1, which indicates that the algorithm was able to learn the differences between the two classes in the training set and generalize them to the validation set. The ROC AUC in the test set was significantly smaller −0.67, meaning that the model may have suffered overfitting. The drop in accuracy and f1-score—see table 8—supports this idea. Table 8 shows the results of an evaluation of the CNN and SVM stack model's performance in the validation and test with the following metrics: accuracy, f1-score, and ROC area under the curve.

TABLE 8

| | VALIDATION | TEST |
|---|---|---|
| ACCURACY | 0.85 | 0.52 |
| F1-SCORE | 0.85 | 0.57 |
| ROC AUC | 1.00 | 0.67 |

However, by adding the information regarding the optical fingerprint to the patient's metadata, the performance of the stratification improved by 5% the ROC AUC in test stage, respectively, in comparison with the SVM model built only with the information provided from patient's metadata. Additionally, there was an increase of 14% in the ROC AUC in the validation stage using the combination of the two sources of information than only using metadata.

Hybrid CNN. The ROC curves of the hybrid CNN are represented in FIGS. 13 and 14. The ROC AUC values are approximately equal in the validation and test, showing that the model did not overfit the training data. By comparing the results of the model with the ones previously discussed, it is possible to conclude that the Hybrid CNN has the best generalization capability since its performance in the test set was the best one. The hybrid CNN achieved an accuracy of 0.72 in the test set, as depicted table 9. This algorithm architecture and the combination of the information of the patient's metadata with the optical fingerprint has improved the performance of the stratification by almost 15% and 10% the f1-score and ROC AUC, respectively, in the test stage, in comparison with the SVM model built only with the information provided from patient's metadata.

Table 9 shows an evaluation of the Hybrid CNN's performance in the validation and test with the following metrics: accuracy, f1-score, and ROC area under the curve (AUC).

| | Validation | Test |
|---|---|---|
| Accuracy | 0.83 | 0.72 |
| F1-score | 0.81 | 0.66 |
| ROC AUC | 0.69 | 0.70 |

An overview of the method of the invention is set out in FIG. 15. In a first step 1510 a fluid sample 9 is obtained from the patient. The fluid sample 9 is generally obtained from a blood sample of the patient and is prepared in step 1515 to obtain a plasma or serum, as described above. A light signal is produced from the laser 1 in step 1520 and the fluid sample 9 is illuminated in step 1530 with the light signal through a lens in the sensing probe. A spectrogram from the fluid sample 9 is acquired in step 1540 and in step 1550 a plurality of spectrogram features from the light signal is extracted.

In step 1555, a plurality of demographic features of comorbidities derived from a patient's health record comparing are also obtained. The extracted plurality of spectrogram features and the plurality of demographic features are then compared with the model in a database to determine a degree of severity of the respiratory disease in step 1560 to output a result in step 1570 which is representative of the degree of severity of the respiratory disease.

The creation of the model in the database is shown in FIG. 16. The plurality of demographic features and the spectrogram features are obtained as outlined above for a group of healthy patients and a group of patients with a respiratory disease in step 1610. In both cases, the features are extracted from the spectrogram created in step 1550 and these are fed to a training system in step 1630 where a learning method is applied in step 1640. The learning method can be one or more of the afore-mentioned learning methods, such as CNN or SVM or a combination thereof.

A verification step was carried out in step 1650 with new data, i.e., spectrogram features and demographic features from a different set of samples.

### REFERENCES

An [1] World Health Organization (WHO): WHO Coronavirus Disease (COVID-19) Dashboard. 2020/10/2. https://covid19.who.int/.
[2] Chen N., Zhou M., Dong X, et al. Epidemiological and clinical characteristics of 99 cases of 2019 novel coronavirus pneumonia in Wuhan, China: a descriptive study. Lancet 2020, 395: 507-13. Doi: 10.1016/S0140-6736(20)30211-7.
[3] Yang J., Zheng Y., Gou X., et al. Prevalence of comorbidities and its effects in patients infected with SARS-CoV-2: a systematic review and meta-analysis. International Journal of Infectious Diseases 2020, 94: 91-95. Doi: 10.1016/j.ijid.2020.03.017
[4] Yang X., Yu Y., Xu J., et al. Clinical course and outcomes of critically ill patients with SARS-CoV-2 pneumonia in Wuhan, China: a single-centered, retrospective, observational study. Lancet Respir Med 2020, 8: 475-81 Doi: 10.1016/S2213-2600(20)30079-5.
[5] Cox M. J., Loman N., Bogaert D., O'Grady J. Co-infections: potentially lethal and unexplored in COVID-19. Lancet Microbe. 2020, 1(1). Doi: 10.1016/S2666-5247(20)30009-4.
[6] Mandell L. A., Wunderink R. G., Anzueto A., et al, Infectious Diseases Society of America/American Thoracic Society Consensus Guidelines on the Management of Community-Acquired Pneumonia in Adults, Clinical Infectious Diseases. 2007, 44: S27-S72. Doi: 10.1086/511159.
[7] National Institute for Health and Care Excellence (NICE). COVID-19 rapid guideline: managing suspected or confirmed pneumonia in adults in the community. 2020. https://www.nice.org.uk/guidance/ng165.
[8] Shi H., Han X., Jiang N. et al. Radiological findings from 81 patients with COVID-19 pneumonia in Wuhan, China: a descriptive study. Lancet Infect Dis. 2020, 20: 425-34. Doi: 10.1016/S1473-3099(20)30086-4.
[9] Bai H. X., Hsieh B., Xiong Z., et al. Performance of Radiologists in Differentiating COVID-19 from Non-COVID-19 Viral Pneumonia at Chest CT. Radiology. 2020, 296: E46-E54. Doi: 10.1148/radiol.2020200823.
[10] Hani C., Trieu, N. H., Saab, I., et al., COVID-19 pneumonia: a review of typical CT findings and differential diagnosis. Diagnostic and Interventional Imaging. 2020, 101: 263-268, Doi: 10.1016/j.diii.2020.03.014.
[11] Centre for Evidence-Based Medicine (CEBM): Differentiating viral from bacterial pneumonia. 2020. https://www.cebm.net/covid-19/differentiating-viral-from-bacterial-pneumonia/.
[12] Mandell L. A., Wunderink R. G., Anzueto A., et al, Infectious Diseases Society of America/American Thoracic Society Consensus Guidelines on the Management of Community-Acquired Pneumonia in Adults, Clinical Infectious Diseases. 2007, 44: S27-S72. Doi: 10.1086/511159.
[13] Gupta D., Agarwal R., Aggarwal A. N., et al. Guidelines for diagnosis and management of community- and hospital-acquired pneumonia in adults: Joint ICS/NCCP(I) recommendations. Lung India. 2012, 29(2): S27-S62. Doi: 10.4103/0970-2113.99248.
[14] World Health Organization (WHO). Use of chest imaging in COVID-19: a rapid advice guide. 2020. https://apps.who.int/iris/handle/10665/332336.
[15] Htun T. P., Sun Y., LanChua H., Pang J. Clinical features for diagnosis of pneumonia among adults in primary care setting: A systematic and meta-review. Scientific Reports. 2019, 9:7600. Doi: 10.1038/s41598-019-44145-y.
[16] Müller B., Harbarth S., Stolz D., et al. Diagnostic and prognostic accuracy of clinical and laboratory parameters in community-acquired pneumonia. BMC Infect Dis. 2007, 2:7-10. Doi: 10.1186/1471-2334-7-10.
[17] Metlay J. P., Waterer G. W., Long A. C., et al. Diagnosis and Treatment of Adults with Community-acquired Pneumonia. American Thoracic society documents. Am J Respir Crit Care Med. 2019, 200 (7): e45-e67. Doi: 10.1164/rccm.201908-1581ST
[18] Marti C., Garin N., Grosgurin O., et al. Prediction of severe community-acquired pneumonia: a systematic review and meta-analysis. Critical Care 2012, 16: R141. Doi: 10.1186/cc 1447.
[19] Cooper G. F., Abraham V., Aliferis C. F., et al. Predicting dire outcomes of patients with community acquired pneumonia. Journal of Biomedical Informatics 2005, 38: 347-366. Doi: 10.1016/j.jbi.2005.02.005.
[20] Zhang S., Zhang K., Yu Y., et al. A new prediction model for assessing the clinical outcomes of ICU patients with community acquired pneumonia: a decision tree analysis. Annals of medicine 2019, 51(1): 41-50. Doi: 10.1080/07853890.2018.1518580
[21] Hashmi M. F., Katiyar S., Keskar A. G., et al. Efficient Pneumonia Detection in Chest Xray Images Using Deep Transfer Learning. Diagnostics 2020, 10: 417. Doi:10.3390/diagnostics10060417
[22] E. Ayan and H. M. Onver. Diagnosis of Pneumonia from Chest X-Ray Images Using Deep Learning. 2019 Scientific Meeting on Electrical-Electronics & Biomedical Engineering and Computer Science (EBBT), 2019: 1-5, doi: 10.1109/EBBT.2019.8741582.
[23] Rahman T., Chowdhury M. E. H., Khandakar A., et al. Transfer Learning with Deep Convolutional Neural Network (CNN) for Pneumonia Detection Using Chest X-ray. Appl. Sci. 2020, 10: 3233. Doi:10.3390/app10093233
[24] Chouhan V., Singh S. K., Khamparia A., et al. A Novel Transfer Learning Based Approach for Pneumonia Detection in Chest X-ray Images. Appl. Sci. 2020, 10: 559. Doi: 10.3390/app10020559.
[25] Sanyaolu A., Okorie C., Marinkovic A., et al. Comorbidity and its Impact on Patients with COVID-19. SN Compr Clin Med 2020:1-8. Doi:10.1007/s42399-020-00363-4.
[26] Gold M. S., Sehayek D., Gabrielli S., et al. COVID-19 and comorbidities: a systematic review and meta-analysis. Postgraduate Medicine, 2020. Doi: 10.1080/00325481.2020.1786964.
[27] Richardson S., Hirsch J. S., Narasimhan M., et al. Presenting Characteristics, Comorbidities, and Outcomes Among 5700 Patients Hospitalized With COVID-19 in the New York City Area. JAMA 2020, 323(20):2052-2059. doi:10.1001/jama.2020.6775.
[28] Guan W. J., Liang W. H., Zhao Y., et al. Comorbidity and its impact on 1590 patients with COVID-19 in China: a nationwide analysis. Eur Respir J 2020, 55(5): 2000547. Doi:10.1183/13993003.00547-2020.
[29] Centers for Disease Control and Prevention (CDC). Coronavirus Disease 2019 (COVID-19): People with Certain Medical Conditions. 2020. https://www.cdc.gov/coronavirus/2019-ncov/need-extra-precautions/people-with-medical-conditions.html.
[30] Zhou B., She J., Wang Y., Ma X. Utility of Ferritin, Procalcitonin, and C-reactive Protein in Severe Patients with 2019 Novel Coronavirus Disease. Research Square 2020. Doi: 10.21203/rs.3.rs-18079/vl.
[31] Qin C., Zhou L., Hu Z., et al. Dysregulation of Immune Response in Patients with Coronavirus 2019 (COVID-19) in Wuhan, China. Clinical Infectious Diseases 2020, 71(15):762-8. Doi: 10.1093/cid/ciaa248.
[32] Ruan Q., Yang K., Wang W., et al. Clinical predictors of mortality due to COVID-19 based on an analysis of data of 150 patients from Wuhan, China. Intensive Care Med 2020, 46(5):846-848. doi:10.1007/s00134-020-05991-x.
[33] Liu T., Zhang J., Yang Y., et al. The role of interleukin-6 in monitoring severe case of coronavirus disease 2019. EMBO Mol Med 2020, 12(7): e12421. doi: 10.15252/emmm.202012421.
[34] Ji D., Zhang D., Xu J., et al. Prediction for Progression Risk in Patients With COVID-19 Pneumonia: The CALL Score. Clinical Infectious Diseases 2020, 71(6): 1393-1399, https://doi.org/10.1093/cid/ciaa414.
[35] Diao B., Wang C., Tan Y., et al. Reduction and Functional Exhaustion of T Cells in Patients with Coronavirus Disease 2019 (COVID-19). MedRxiv 2020. Doi: 10.3389/fimmu.2020.00827
[36] Zhang L., Yan X., Fan Q., et al. D-dimer levels on admission to predict in-hospital mortality in patients with Covid-19. J Thromb Haemost 2020,18(6):1324-1329. doi: 10.1111/jth.14859.
[37] Wynants L., Calster B. V., Collins G. S., et al. Prediction models for diagnosis and prognosis of covid-19: systematic review and critical appraisal. BMJ 2020, 369:m1328. doi: 10.1136/bmj.m1328.
[38] Gong J., Ou J., Qiu X., et al. A Tool for Early Prediction of Severe Coronavirus Disease 2019 (COVID-19): A Multicenter Study Using the Risk Nomogram in Wuhan and Guangdong, China. Clin Infect Dis. 2020,71(15):833-840. doi:10.1093/cid/ciaa443.
[39] Zhu J. S, Ge P., Jiang C., et al. Deep-leaming artificial intelligence analysis of clinical variables predicts mortality in COVID-19 patients. ACEP Open 2020:1-10. Doi: 10.1002/emp2.12205.
[40] Fang C., Bai S., Chen Q., et al. Deep learning for predicting COVID-19 malignant progression. MedRxiv 2020. doi: 10.1101/2020.03.20.20037325.
[41] Wang S., Zha Y., Li W. A fully automatic deep learning system for COVID-19 diagnostic and prognostic analysis. Eur Respir J 2020, 56: 2000775 Doi: 10.1183/13993003.00775-2020.

### REFERENCE NUMERALS

610 Input Layer
620a-d Encoding block
630 Tensor
640 Fully connected layer
650 Output
700 Alternative architecture
710 CNN output probability
720 Metadata feature
730 Support vector machine
820a-d Encoding blocks
830 Fully connected block
840 Non-spectral feature set
850 Metadata feature set
860 Full connected layer
870 Layer
880 Dense layer
890 Output

1. A method for stratifying a patient infected with a respiratory disease comprising: providing (1510)
a fluid sample (9) from the patient:
producing (1520) a light signal from a laser (1);
illuminating (1530) the fluid sample (9) with the light signal through a lens in a sensing probe (8);
acquiring (1540) a spectrogram from the fluid sample (9);
extracting (1550) a plurality of spectrogram features from the light signal; comparing (1560) the extracted plurality of spectrogram features with a model in a database to determine a degree of severity of the respiratory disease; and outputting (1570) a result.

2. The method of claim 1, wherein the fluid sample (9) is one of a plasma sample or a serum sample.

3. The method of claim 1, wherein the extracting (1550) of the plurality of features comprises extraction of time features and frequency derived features.

4. The method of claim 1 further comprising providing (1555) of demographic features of comorbidities derived from a patient's health record and comparing (1560) both the demographic features and the spectrogram features with the model to determine the degree of severity of the respiratory disease.

5. The method of claim 1, wherein the model is a combination one or more of a support vector machine (SVM), k nearest neighbors, or random forests, and a convolutional neural network (CNN) model.

6. The method of claim 1, further comprising modulating (110) the light signal from the laser (I).

7. The method of claim 1, wherein the extraction (138) of the plurality of spectrogram features in the light signal is carried out over periods of time.

8. The method of claim 1, wherein the model is created by one of a supervised learning method, for example a support vector machine, k nearest neighbors, or random forests, or an unsupervised learning method, for example a clustering algorithm, or a regression model.

9. The method of claim 1, wherein the respiratory disease is a viral disease.

10. A device for stratifying a patient infected with a respiratory disease comprising:
a laser (1) connected through an optical fiber with a sensing probe (8) with a microlens for illuminating a fluid sample (9) from the patient;
a detector (16) for acquiring (130) a spectrogram from the sample (9);
a temperature measurement device, and
a computer (17) adapted to analyze the spectrogram, extract (1550) spectrogram features from the spectrogram, compare (1560) the extracted spectrogram features with stored features in a model and output (1570) a result of the degree of severity of the respiratory disease.

11. The device of claim 10, wherein the sensing probe (8) comprises a microlens at the end of the optical fiber.

12. The device of claim 10, wherein the computer (17) is further adapted to obtain demographic features of comorbidities derived from a patient's health record and compare (1560) both the demographic features and the spectrogram features with the model to determine the degree of severity of the respiratory disease.

13. The device of claim 10, wherein the model is a combination of one or more of a support vector machine (SVM), k nearest neighbors, or random forests, and a convolutional neural network (CNN) model.

14. The device of claim 10, wherein the respiratory disease is a viral disease

15. A method for creation of a model for stratifying a patient infected with a respiratory disease, the creation of the model using a plurality of spectrogram features from a light signal and a plurality of demographic features from the patient health record, the method comprising:
producing (1520) a light signal from a laser (1);
illuminating (1530) with the light signal through a microlens in a sensing probe (8) a series of fluid samples (9) of healthy and diseased patients with known comorbidities and health outcomes;
acquiring (1540) a spectrogram from the fluid sample (9);
extracting (1550) the plurality of spectrogram features from the light signal; entering (1555) the plurality of demographic features and health outcomes; and applying a learning method to the extracted plurality of spectrogram features and the entered plurality of demographic features to correlate the extracted plurality of spectrogram features and the entered plurality of demographic features with the health outcomes to create the model in a database.

16. The method of claim 15, wherein the learning method is at least one of a supervised learning method, such as a support vector machine, an unsupervised learning method, such as clustering algorithms, or a regression model.

17. The method of claim 15, wherein the applying of the learning method comprises a training a convolutional neural network using the spectrogram features and then training a support vector machine using an output of the trained convolutional neural network and the demographic features.

18. The method of claim 13 wherein the applying of the learning method comprises a training of a first set of encoding blocks of a convolutional neural network with the spectrogram features and then further training the convolutional neural network with the demographic features.

19. The method of claim 16 further comprising using time and frequency features of the light signal in the learning method.

20. The method of claim 16, wherein the respiratory disease is a viral disease.
